(19) United States
(12) Patent Application Publication
(10) Pub. No.: US 2020/0309753 A1
(43) Pub. Date: Oct. 1, 2020

(54) SYSTEMS AND METHODS FOR MONITORING MICROBIOME MARKERS/MOLECULES IN WASTE ENVIRONMENTS

(71) Applicant: Egesta Technologies, LLC, League City, TX (US)

(72) Inventors: Madonna Mamerow, Galveston, TX (US); Stephen Davila, Pearland, TX (US)

(21) Appl. No.: 16/833,569

(22) Filed: Mar. 28, 2020

Related U.S. Application Data
(60) Provisional application No. 62/826,485, filed on Mar. 29, 2019.

(51) Int. Cl. G01N 33/00; G01N 33/497; G01N 30/72

(52) U.S. Cl. CPC G01N 33/0031; G01N 33/497; G01N 2030/025; G01N 2033/4975; G01N 30/722

(57) ABSTRACT

A gas collection and analysis system for detecting microbiomes is described. The system includes a waste disposal site, a sorbent cartridge coupled adjacent to the waste disposal site, wherein the sorbent cartridge includes a sorbent media configured to absorb a gas sample from within the waste disposal site, a sorbent heating block configured to heat the sorbent cartridge and release the gas sample absorbed by the sorbent media, and a pumping system coupling the sorbent cartridge to an ionized gas analyzer mass spectrometer, the pumping system configured to direct the flow of the released gas sample from the sorbent media to the gas analyzer mass spectrometer for chemical analysis. Methods for detecting microbiomes of a user are also described herein.

### RELATED APPLICATION(S)

The present application claims priority from and the benefit of U.S. Provisional Application Ser. No. 62/826,485, filed Mar. 29, 2019, the disclosure of which is hereby incorporated herein in its entirety.

### FIELD

The present invention is directed generally to systems and methods for detecting and tracking gut microbiome volatile organic compounds.

### BACKGROUND

The thought of being “gassed” often invokes images of gas masks being frantically donned to protect from inhalation injury, but the fact is, we are gassed daily from within our bodies through the byproducts of microbial colonization, metabolism and fermentation as well as various organ and enzymatic processes. Microbes residing in the gastrointestinal (GI) tract not only play a major role in producing gases, but also in consuming them. Gases are the smallest bioactive molecules utilized for sustaining life and can easily and rapidly traverse cell membranes without the need of binding to specific cell membrane receptors. Once gases enter a cell they can interact with intracellular enzymes and ion channels and can cause post-translational modifications to proteins creating genetic changes.

Among the most abundant intraluminal gases are carbon dioxide (CO₂), hydrogen (H₂), methane (CH₄), hydrogen sulfide (H₂S), nitrogen (N₂), oxygen (O₂) nitric oxide (NO), ammonia (NH₃), carbon monoxide (CO) and short-chain fatty acids (SCFA). These “gasotransmitters” have been shown to act as mediators and modulators along with other trace volatile organic compounds (VOCs) in the bidirectional communication between the gut, immune system, and brain, which is known as the “microbiota-gut-brain axis.” These gases also serve as nutrients for various tissues, such as nerve cells. Thus, alterations in the gut microbiome (GM) balance, whether through diet, stress, radiation exposure, antibiotic or probiotic usage, disease, infection, or confinement can alter the production of gasotransmitters and cause direct functional consequences to host health, creating a very insidious type of biological threat. Some diseases and disorders reporting alterations in metabolic gas production include GI cancers, mental illness, diabetes, and infertility to name a few.

Optimizing gasotransmitter pools through diet and/or symbiotic microbes may prove beneficial for protecting the microbiome, brain and immune function and preventing opportunistic infections. Thus, accurately and quickly measuring the GM gasotransmitters (GM VOCs) may provide benefits to overall human health. However, there may be a need for improving how GM VOC analysis is performed in various settings. Improvements may be needed in the context of confined spaces, such as on cruise ships, space crafts, and space stations. Improvements are also needed in order to produce a commercially viable GM VOC analyzer that is affordable for hospitals, clinics, and the average consumer in the home-setting.

For spaceflight, gas analyzers must be low weight, low power consuming, easy to operate, take little storage space, and use limited consumables, which is also beneficial for commercial product development on Earth. However, many of the rapid trace VOC detection methods and devices are large devices with long analysis times that require multiple sample preparation steps. Other faster and smaller trace gas analyzers have low detection accuracy and reliability due to the decreased resolution. These devices also do not offer real-time sample collection methods needed for GM VOC detection. Accurate calibration is also a concern when environmental changes occur or when the environment is unknown, such as with temperature, humidity, gas volume, flow rates, and/or distance from the gas source. Many gas analyzers are also not easily calibrated or operated by laypersons and untrained users making trace chemical analysis expensive and difficult for an at-home user, increasing errors in results and taking extended periods of time to operate. In the professional setting, time is extremely valuable not only for the delivery of results, but also for the personnel involved including astronauts, military and ship personnel and within hospitals, clinics, and labs. Some gas sensor systems are also known to be affected by small variations in CO₂ and water in the environment which would not be feasible in the high CO₂ environment of a spacecraft or in humid environments on cruise ships or in lavatory facilities where GM VOC analysis may take place. Other systems may only be capable of detecting a few VOCs and/or unable to detect trace levels of VOCs.

### SUMMARY

Embodiments of the invention are directed to methods, systems and devices for detecting and tracking GM VOCs. The present invention represents a gas analyzer for use in the vicinity of a toilet, toilet accessory, or device that captures waste from organisms. There is no need to directly analyze waste material as the sensitivity of the instrument ranges from part per billion to part per thousand.

An embodiment of the present invention is directed to a gas collection and analysis system for detecting microbiomes. The system may include a waste disposal site, a sorbent cartridge coupled adjacent to the waste disposal site, wherein the sorbent cartridge includes a sorbent media configured to absorb a gas sample from within the waste disposal site, a sorbent heating block configured to heat the sorbent cartridge and release the gas sample absorbed by the sorbent media, and a pumping system coupling the sorbent cartridge to an ionized gas analyzer mass spectrometer, the pumping system configured to direct the flow of the released gas sample from the sorbent media to the gas analyzer mass spectrometer for chemical analysis.

In some embodiments, the system may further include one or more sensors coupled to the waste disposal site configured to control the pumping system and heating of the sorbent heating block.

In some embodiments, the ionized gas analyzer mass spectrometer includes a quadrupole, an ion trap, a time of flight mass spectrometer, an ion mobility, a differential mobility, or a field asymmetric ion mobility spectrometer.

In some embodiments, the system may further include a controller configured to process data of the ionized gas analyzer mass spectrometer and/or concentration of absorbed and releases gases in real-time and over period of time.

In some embodiments, the ion mobility measurement may be done by IMS, DMS, FAIMS, or hyphenated techniques of ion mobility and mass spectrometry with gas chromatography.

In some embodiments, the ionization mechanism includes nickel-63 foil, electron ionization, corona discharge, chemical ionization, photoionization, electro-spray, atmospheric pressure chemical ionization, thermal ionization, DART, desorption electro spray, or metal spray.

In some embodiments, the sorbent cartridge is reusable, thereby allowing the system to obtain gas samples of multiple, different users.

In some embodiments, the sorbent cartridge may include a carboxen sorbent, a liquid sorbent, or one or more membranes.

In some embodiments, the sorbent cartridge may include a single or multiple sorbent media for analysis.

In some embodiments, the waste disposal site is a toilet.

In some embodiments, the waste disposal site is a waste collection bag.

In some embodiments, the sorbent cartridge may be configured to be attached to a disposable mouthpiece to obtain the gas sample from a breath analysis.

In some embodiments, the gas sample may be utilized for online analysis via gas chromatography.

In some embodiments, the ionized gas analyzer mass spectrometer utilizes gas chromatography.

In some embodiments, the system may be configured to monitor and track the gas samples of a user for evaluation.

Another embodiment of the present invention is directed to a method for detecting microbiomes of a user. The method may include providing a gas collection and analysis system comprising a waste disposal site, wherein the waste disposal site, a sorbent cartridge coupled adjacent to the waste disposal site, wherein the sorbent cartridge includes a sorbent media configured to absorb a gas sample from within the waste disposal site, a sorbent heating block configured to heat the sorbent cartridge and release the gas sample absorbed by the sorbent media, and a pumping system coupling the sorbent cartridge to an ionized gas analyzer mass spectrometer, the pumping system configured to direct the flow of the released gas sample from the sorbent media to the gas analyzer mass spectrometer for chemical analysis; having the user sit on the waste disposal site; absorbing a gas sample of the user from the waste disposal site into the sorbent cartridge; having the user stand up from the waste disposal site; heating the sorbet cartridge to release a gas sample from the sorbent cartridge; pumping the released gas sample from the sorbent cartridge to the gas analyzer mass spectrometer; determining the chemical compounds in the gas sample based on a chemical analysis from the gas analyzer; and providing the user health data based on the chemical analysis.

In some embodiments, the method may include removing the sorbent cartridge from the waste disposal site for analysis at a remote location.

In some embodiments, the gas collection and analysis system may further include one or more sensors coupled to the waste disposal site, the sensors being configured to control the pumping system and heating of the sorbent heating bloc when the user stands up from the waste disposal site.

In some embodiments, the gas collection and analysis system may further include a controller configured to process data of the ionized gas analyzer mass spectrometer and/or concentration of absorbed and releases gases to the user in real-time and over period of time.

In some embodiments, the sorbent cartridge is reusable, thereby allowing the system to obtain gas samples of multiple, different users.

Some embodiments of the present invention are directed to Earth-based systems, methods, and devices.

Some embodiments of the present invention are directed to space-based systems, methods, and devices.

Some embodiments include a modular inlet system that can direct, optionally vacuum, air from the headspace of a toilet, preconcentrate the GM VOCs on a sorbent media housed within a sorbent cartridge, and release the gases into the detection chamber of either a local or remote gas analyzer for analysis. An embodiment for spaceflight purposes may also include a waste collection bag with membrane port to avoid particulate contamination and a sampling line directing airflow to the sorbent cartridge inlet system. Whilst another embodiment allows for the sorbent cartridge (using a sorbent tube configuration) to be used alone as a gas collection device for capturing breath samples as an indirect measure of the GM VOCs and then deposited into the heated block for thermal desorption and analysis.

Machine learning methods (artificial intelligence or AI) can be applied to identify patterns from the data in real-time that correspond to health diagnosis information. The invention may be applied as a health screening and health tracking device (i.e., early detection of infections, viruses, cancer, ingested toxins); to monitor fertility (i.e., ovulation and menstrual changes); and to monitor/study the health of humans living in confined spaces, extreme environments, or within vulnerable populations where infections can spread rapidly (i.e., ships, space stations, military bases, hospitals, long-term care facilities).

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1A illustrates a sorbent cartridge in a cylindrical tube configuration with air flow being directed through a packed sorbent media according to embodiments of the present invention.

FIG. 1B illustrates a sorbent cartridge with sorbent material inside the cartridge according to embodiments of the present invention.

FIG. 1C illustrates a sorbent cartridge with a sorbent media coating the interior surface of the cartridge and airflow directed over the surface of the sorbent material according to embodiments of the present invention.

FIG. 1D illustrates a sorbent cartridge with a disposable mouthpiece attachment for collection of breath samples as an alternative indirect measurement of gut microbiome volatile organic compounds according to embodiments of the present invention.

FIG. 2A illustrates an ambient (not-heated) gas pre-concentration inlet system with a removable sorbent cartridge connected to the back of a standard toilet seat according to embodiments of the present invention.

FIG. 2B illustrates a thermal (heated) desorption gas pre-concentration inlet system connected to a standard toilet seat according to embodiments of the present invention.

FIG. 3A illustrates a removable sorbent cartridge attached to a toilet according to embodiments of the present invention.

FIG. 3B illustrates the sorbent cartridge removed from the toilet of FIG. 3A and coupled to a preconcentration inlet of the chemical analyzer according to embodiments.

FIG. 4 illustrates a heated transfer line connected to the output of the sorbent cartridge to the input of a chemical analyzer according to embodiments of the present invention.

FIG. 5 illustrates a waste collection bag connected to a chemical analyzer according to embodiments of the present invention.

FIG. 6 illustrates the thermal desorption gas pre-concentration inlet system of FIG. 2B connected to a waste containment bag and membrane filter according to embodiments of the present invention.

FIG. 7 illustrates a pre-concentration thermal desorption gas analysis system according to embodiments of the present invention.

FIG. 8 illustrates an exemplary configuration of a mass spectrometer (swab) inlet/transfer line connection according to embodiments of the present invention.

FIG. 9 illustrates an exemplary system according to embodiments of the present invention.

FIG. 10 illustrates an exemplary system according to embodiments of the present invention.

FIG. 11 is a graph illustrating the normalized response of acetone peaks when analyzed against a calibration curve according to embodiments of the present invention.

FIG. 12A-12B are graphs illustrating IMS Plasmogram (A) versus Mass Spectrum (B).

FIGS. 13A-13B are graphs illustrating negative (A) and positive (B) modes of detection.

FIG. 14 is a flow diagram illustrating an exemplary use of the system according to embodiments of the present invention.

### DETAILED DESCRIPTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

The present invention now will be described more fully hereinafter with reference to the accompanying drawings, in which illustrative embodiments of the invention are shown.

In the figures, certain layers, components or features may be exaggerated for clarity, and broken lines illustrate optional features or operations unless specified otherwise. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. The sequence of operations (or steps) is not limited to the order presented in the claims or figures unless specifically indicated otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

The following descriptions are example embodiments of the present invention. The system of the present invention may be generally referred to herein as “Egest™.”

Embodiments of the invention may provide a basis to monitor and track an individual's health via gases and log irregularities due to changes in their gaseous profile due to illness or other complications in their biology. Target gases include, carbon dioxide (CO₂), hydrogen (H₂), methane (CH₄), hydrogen sulfide (H₂S), nitrogen (N₂), oxygen (O₂) nitric oxide (NO), ammonia (NH₃), carbon monoxide (CO) and short-chain fatty acids (SCFA) as well as trace VOCs from various compartments of the human volatolome (e.g., feces, urine, blood, saliva, perspiration, milk, emesis, flatus, breath).

Pursuant to embodiments of the present invention, a gas collection and analysis system for use in or near the vicinity of a waste collection system for the gas analysis and detection of microbiomes is provided. The gas collection and analysis system may allow the detection of numerous trace GM VOCs in various environmental conditions. Methods of using a gas collection and analysis system are also provided.

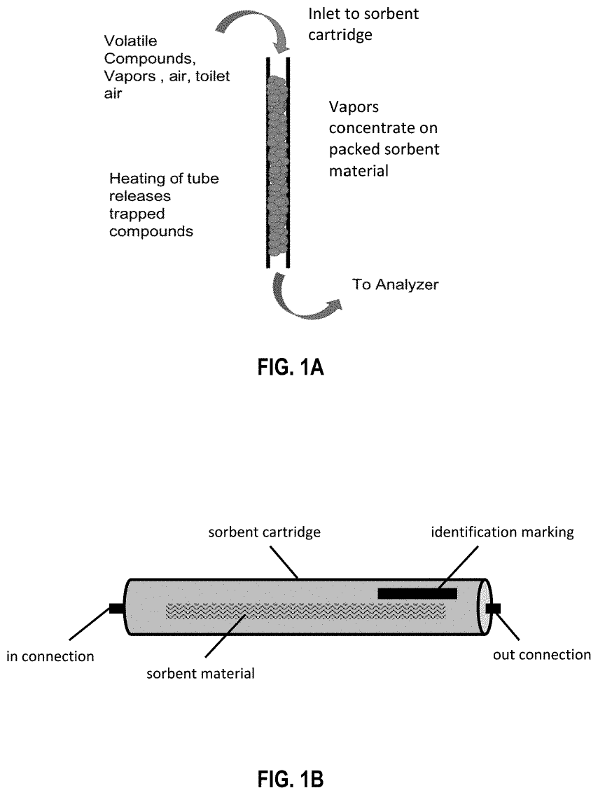

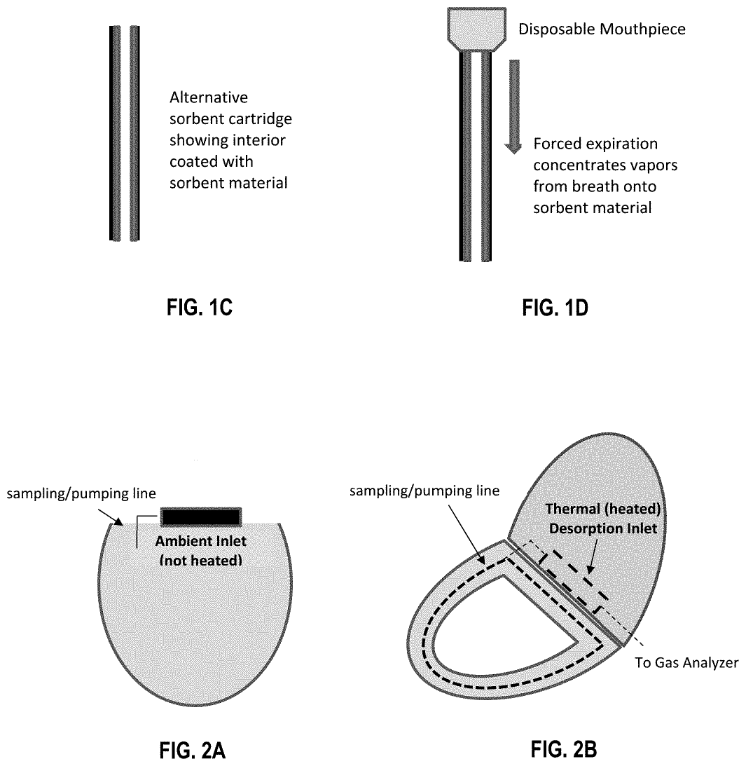

In some embodiments, the present invention may use a conventional sample collection procedure known as thermal desorption for volatile and semi volatile compounds. Collection of these volatile and semi-volatile compounds into sorbent cartridges (see, e.g., FIGS. 1A-1D) is ideal for trace-level analysis of GM VOCs. Thermal desorption is the process of collecting and desorbing analytes from a sorbent media using heat and a flow of gas. The released gas is focused into the analyzer MS or IMS or hyphenated gas chromatogram resulting in symmetric narrow peaks. Other advantages to thermal desorption may include lowering the limits of detection and allowing water or liquids to be purged from samples, thereby increasing the selectivity of the sorbent phase. Thermal desorption also does not require post-processing of samples and when air is used as the carrier does not need large tanks of compressed gas for calibration or operation.

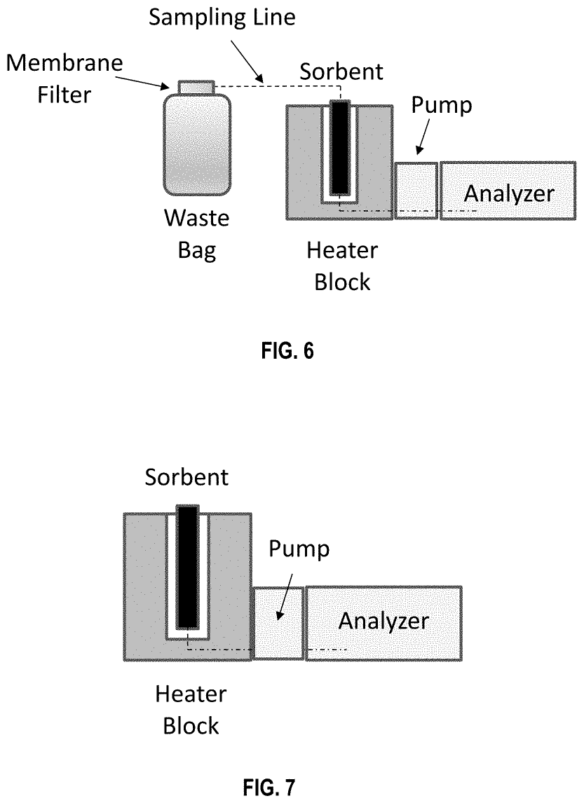

GM VOCs produced during the waste elimination process concentrate on a preconcentration medium (sorbent media) housed within a sorbent preconcentration container (sorbent cartridge) that is integrated into or near a toilet seat. Note that the present invention does not limit the sorbent cartridge to strictly toilets. For example, in some embodiments, the present invention may be used with waste collection bags and breath tubes through a valve assembly and pump or disposable mouthpiece, respectively (see, e.g., FIG. 1D, FIG. 5, FIG. 6).

Preconcentration sorbent cartridges allow for large volumes of air to pass over or through a sorbent media to collect and concentrate the emitted VOCs for analysis. This may be beneficial for the present invention because the length of time spent using the lavatory facility varies and the GM VOCs may become diluted by air if sampled directly. Therefore, concentrating the GM VOCs may result in acquiring more sample to be delivered into the detection chamber of the gas analyzer at once, using a conventional thermal desorption process which can provide greater sensitivity and specificity during analysis and avoids losing VOCs that may be of low concentration.

In some embodiments, the sorbent material may comprise of one or more sorbents which may allow it to capture a variety of VOCs with varying chemical characteristics (i.e., size, charge, boiling point, etc.). For example, in some embodiments, the sorbent media may be comprised of C2-C26, Tenax, graphitized carbon, Carbopack, Carbosieves, Carboxen, and/or liquid polymer. In some embodiments, the sorbents may be suitable for collecting and/or analyzing biosamples of an organism, human, or animal including urine, feces, saliva, breath, sputum and the like, that will not irreversibly bind the VOCs of interest and will release the VOCs collected for analysis by a gas analyzer.

Referring to FIGS. 1A-1D, a sorbent pre-concentration container (e.g., sorbent cartridge) according to embodiments of the present invention is illustrated. The sorbent cartridge may comprise a tube or other enclosed shape coated with, or housing a material coated with, a sorbent media (or material). For example, as shown in FIGS. 1A-1B, an embodiment of a sorbent cartridge in a cylindrical tube configuration with air flow being directed through a packed sorbent media. As shown in FIGS. 1A-1C, the sorbent cartridge may comprise a sorbent media coating the interior surface of the cartridge and airflow directed over the surface of the sorbent material. The sorbent cartridge (sorbent media) is configured to absorb gases onto the surface for analysis and biological tracking of a subject's waste VOCs at a collection point or disposal site.

In some embodiments, the sorbent cartridge may enclose the sorbent material within an interior surface of the cartridge such that the sorbent material cannot be touched, capture oil or other contaminants from users' fingers or other surfaces. In some embodiments, the sorbent cartridge may be made of stainless steel, aluminum, glass or other material that can sustain high temperatures (up to 300° C.) and that does not out-gas or introduce contaminates into the sample collection when the cartridge is heated. The sorbent cartridges of the present invention are a special device that is different than a typical carbon filter that is only used to purify the air. Conventional filters have exposed carbon which also allows the user to touch the carbon during installation, which may leave oil residues on the filter from the skin-giving false results. Conventional filters also cannot typically be heated to a desired level (i.e., about 150° C. or higher) as the sorbent cartridge is formed of plastic material that, even if capable of heating, can off-gas its own VOCs once heated which can skew data. The carbon types typically used in conventional filters will most likely irreversibly bind chemicals given the purpose of prior filters is commonly to absorb odors, thus if it were capable of heating it would not release the captured chemicals into the system for analysis.

In order to decrease sample collection and analysis time to achieve a rapid analysis of a collected sample, in some embodiments, the size of the sorbent cartridge orifice is configured such that the cartridge may capture the greatest quantity of VOCs possible and also allow the VOCs to be released quickly and efficiently for analysis using a conventional thermal desorption process.

In some embodiments, the sorbent cartridge may be configured to be removed and analyzed by a gas analyzer at a different location. In some embodiments, the sorbent cartridge may be configured such that the cartridge is directly connected to the heated inlet of the gas analyzer.

Referring to FIGS. 2A-10, a gas collection and analysis system for the gas analysis and detection of microbiomes according to embodiments of the present invention is illustrated. In some embodiments, the system may be used in or near the vicinity of a waste collection. In some embodiments, the sorbent cartridge described herein may be inserted into an inlet compartment of the system through an easy load and lock mechanism or push-spring mechanism where air is pulled across the sorbent medium. The pulling of the air sample into the sorbent cartridge is accomplished via a flow system operated via a pump pulling as little as 0.1 mL/min up to 5 L/min, which is housed within the inlet compartment or coupled to a gas analyzer. In some embodiments, the flow system may have a sampling line connected to the waste collection site (e.g., toilet seat, toilet bowl headspace, or other waste collection point) or the sorbent cartridge may be open on one end with no sampling line depending on the proximity to the gas source and connected to the pump at the opposite end of the cartridge. In some embodiments, the sampling line may comprise PEEK, stainless, glass or other material that does not absorb or out-gas volatiles that could contaminate the sample.

In some embodiments, the system can be turned on manually or automatically. For example, in some embodiments, the system may comprise a pressure sensor in the toilet seat (e.g., in the rubber grips of the seat), or coupled to the toilet and/or the toilet seat which can detect when a user is seated. In some embodiments, when the system senses a user on the toilet, the pump may be activated to automatically begin evacuating the air from the headspace of the waste collector (e.g., toilet or other) and through the sorbent cartridge. As air is evacuated from the waste collector, waste VOCs may be collected or trapped within the sorbent media and the air vented. In some embodiments, when the user stands, the (pressure) sensors (in the seat) may direct the system to end the sample preconcentration collection process (optionally closing/opening any valves). In some embodiments, the system may use a light sensor, body heat sensor, opto-electronic sensor, camera or proximity sensor or combinations of the same.

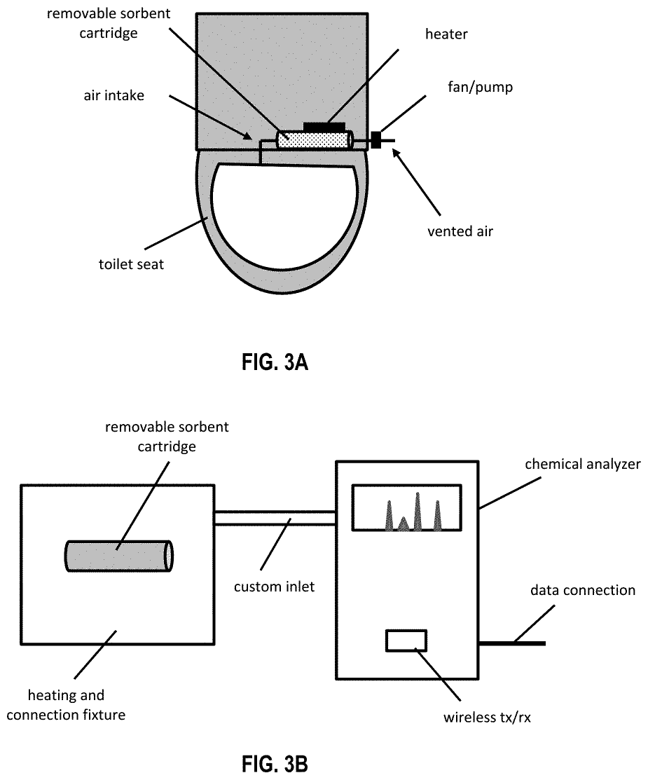

In some embodiments of the present invention, the sorbent cartridge may be configured to be removed from an ambient (not-heated) inlet compartment, and placed in a biohazard package, for example, if the cartridge is transported to an alternate site for analysis. An ambient (not-heated) gas preconcentration inlet system with removable sorbent cartridge connected to the back of a standard toilet seat according to embodiments of the present invention is illustrated in FIGS. 3A-3B.

When transferred to an alternate site for analysis, the sorbent cartridge may be inserted into a thermal desorption (heated) inlet which is coupled to a gas analyzer. The thermal desorption inlet allows the gas molecules to be released from the sorbent medium for sample analysis. Benefits of this embodiment may include reduced cost to the user related to not needing to purchase a chemical analyzer. This may also allow for batch analysis of sorbent cartridges to be performed for multiple users, multiple sorbent cartridges with only one chemical analyzer that is located at a central site such as a core laboratory within a hospital, academic institution, military base, ship, space station, etc.

In some embodiments, the system of the present invention may house the sorbent cartridge within a heater mantle (e.g., heater block) located at the waste disposal site where the inlet system directly interfaces with the chemical analyzer of choice. For example, a preconcentration thermal (heated) desorption gas inlet system connected to a standard toilet seat according to embodiments of the present invention is illustrated in FIG. 2B. In some embodiments, system may comprise a metal block/sleeve made of stainless steel, aluminum, copper, and/or mixtures of the above. In some embodiments, when the sample collection process is ended (e.g., determined by the sensors described herein) the heater mantle may begin to increase the temperature according to a pre-set temperature profile to desorb the sample into the gas analyzer. In some embodiments, the pre-set temperature may range from ambient to about 150° C. In some embodiments, the released gases from the cartridge may be vacuumed into a gas analyzer for analysis. This allows the system to have hands-free operation, thereby allowing for decreased sample contamination and real-time results.

As shown in FIG. 2A and FIG. 2B, in some embodiments, the system (e.g., cartridge) may be attached at back of the toilet seat (or other location) for ease of access. Alternatively, the inlet compartment of the system can be placed on the side or under the tank, or on the base of the toilet (or other waste collection point). This may allow for easy removal of the sorbent cartridge when the sorbent cartridge is deposited into a heated inlet coupled to a gas analyzer for analysis that is not directly connected to the sampling system or is at a different location, for example, as shown in FIG. 3B.

In some embodiments, the sorbent cartridge is not coupled to a toilet/toilet seat, but rather, is connected at one end to a disposable mouthpiece to capture VOCs from the breath during a forced expiration. As shown in FIG. 1D, in some embodiments, the sorbent cartridge with a disposable mouthpiece attachment for collection of breath samples as an indirect measurement of GM VOCs (not involving air from the toilet). In some embodiments, the mouthpiece is removable, which allows the sorbent cartridge to be coupled to the thermal desorption inlet of a gas analyzer. The mouthpiece may allow for indirect measurements of GM VOCs without the need for a new gas inlet or analyzer.

Referring to FIGS. 5 and 6, in some embodiments, the system may comprise a waste containment bag and membrane filter. The waste containment bag and membrane filter may be connected to a sampling line which directs VOCs into a sorbent cartridge preconcentration via a pump. The pump may be coupled to a thermal desorption inlet and gas analyzer at the site of the waste disposal. In some embodiments, this alternative connection for the system may be used for spaceflight purposes. This alternative connection may allow for pre-existing methods of waste collection during spaceflight to be maintained, i.e., without the use of a novel/customized toilet or toilet seat. Instead, the waste containment bag may be used with a thermal desorption inlet that draws the chemical sample from the bag and desorbs the concentrated gas sample into the chemical analyzer.

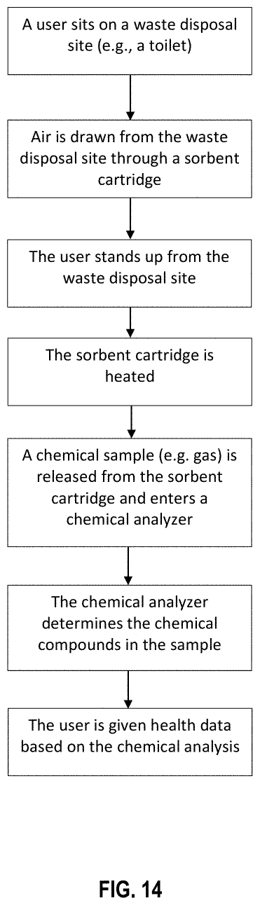

Exemplary methods of using the gas collection and analysis system according to embodiments of the present invention are also provided. For example, as shown in FIG. 14, a method may include (1) a user sitting on a waste disposal site (e.g., a toilet); (2) drawing air from the waste disposal site through a sorbent cartridge; (3) the user standing up from the waste disposal site; (4) heating the sorbent cartridge; (5) collecting a chemical sample released from the heated sorbent cartridge and entering the sample into a chemical analyzer; (6) determining the chemical compounds within the sample; and (7) providing health data to the user based on the chemical analysis of the sample.

In some embodiments, analysis of the collected sample begins by heating the sorbent medium to release the gas molecules. In some embodiments, operations of the system may be controlled (e.g., programmatically) such as operating a heater and controlling actuation of any valves or vacuum pumps, as well as modifications to a user through an interface for selectable settings. The temperature profile may control heating of the sorbent, and molecules desorb according to their affinity with the sorbent. This affinity is similar to a molecule's boiling point, with low boiling small molecules desorbing first, then higher boiling molecules desorbing as the temperature rises. In some embodiments, resistive heating may be used with thermocouples to measure the temperature of the heater. The heating of the sample may be induction through a heater cartridge. In some embodiments, a small metal (e.g., aluminum) heat block/sleeve may be used. The heat block/sleeve may extend over at least a majority, typically at least 80%, or all the length of the sorbent cartridge. In some embodiments, the toilet seat may be insulated/heat shielded to protect the user for burn injury. In some embodiments, heating of the sample may be controlled to initiate only after the user stands. In some embodiments, the heat block may use a lever/latch push and lock mechanism to load the sorbent cartridge, for example, similar to the unheated ambient version described herein. It may be heated from ambient to about 150° C. to release the volatiles collected in the sorbent material.

In some embodiments, the released gaseous molecules are pulled or pushed into an ionization region for analysis. The ionization region is a region where the gaseous molecules become ions. The gaseous molecules become ions through ionization and are charged molecules with a positive or negative charge. A multitude of sources may be used to form ions and can vary from instrument to instrument, some of the most common types applicable in this analysis. In some embodiments, the ion sources useful in the formation of ions for analysis may include radioactive nickel-63 foil, electron ionization, corona discharge, chemical ionization, photoionization, electrospray, atmospheric pressure chemical ionization, thermal ionization, DART, desorption electrospray, or metal spray.

The gas analysis for the present invention occurs via mass spectrometry (quadrupole, ion trap, time of flight). If a mass spectrometer is not available, another type of analyzer that may be used with the present invention includes ion mobility and analysis occurs based on the mobility of an ion through a buffer gas. In some embodiments, the ion mobility measurement may be done via IMS, DMS, FAIMS, or hyphenated techniques of ion mobility and mass spectrometry. In some embodiments, hyphenated techniques may include IMS-MS, GC-MS, GC-IMS to analyze directly or from sorbent materials where the gas is collected at waste collection point (i.e., toilet). Alternatively, in some embodiments, the analyzer may be separate from the waste collection, and a sorbent cartridge used to collect and tag sampling for analysis at the instrument at a different location.

In some embodiments, following analysis of a sample, the sorbent cartridges may be reusable. This may allow for reducing consumables cost. In some embodiments, to clean and sterilize the sorbent cartridge, a bake-out program may b used to heat the cartridge while in the thermal desorption inlet (heater block) to a high temperature for a predetermined amount of time (e.g., about 250° C. to about 300° C. for about 10 minutes). Thus, a user may not be required to change the sorbent cartridge out each time after a user eliminates waste at the waste collection site where a thermal desorption inlet is incorporated, thereby allowing the sorbent cartridge to be multi-use rather than single use-disposable.

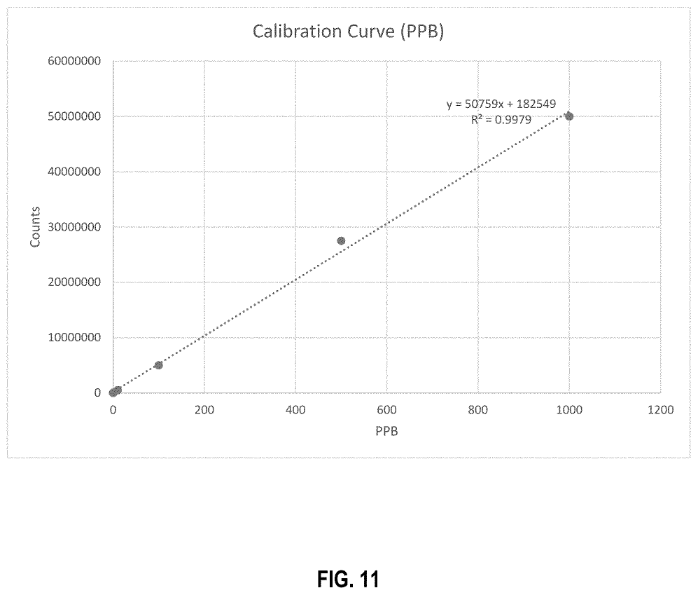

Referring to FIG. 11, data interpretation may be performed through the use of calibration curves and sample quantitation is possible allowing for tracking and recording of biological statistics. FIG. 11 illustrates the normalized response of acetone peaks when analyzed against a calibration curve and the amount of signal coming from the preconcentrator can be calculated.

In some embodiments, the data provided by the chemical analyzer can be used in a variety of ways. In some embodiments, AI (machine learning) may be used to determine correlations with diet/eating habits, environmental factors, behavioral data, and the VOC patterns discovered by the invention. Independent of how the correlation data is generated, the information generated may be used to track and/or adjust diet/eating habits, for tracking historical obesity data, geographical mapping of disease incidence, food imports, domestic crops, pesticides, etc. for health assessments and screenings.

In some embodiments, the system of the present invention may be installed on a toilet seat at a user's home, hospital, clinic, nursing home or other patient facility, as well as on ships, military bases or other location where health monitoring may be needed for screening and prevention of infectious disease or other illness. In some embodiments, the system may be used for academic research purposes and clinical studies.

In some embodiments, the system of the present invention may be used at the International Space Station (ISS) or kept at a home base for real-time testing with astronauts before, during, and after spaceflights. The system may be used to compare collected data with metabolomics data from the Astronaut Microbiome Project. Further embodiments may include developing unique software for the system of the present invention using algorithms to perform artificial intelligence (AI) machine learning of VOC patterns and determine correlations with diet/eating habits, environmental factors, and behavioral data onboard the ISS. These studies may help determine how VOC levels correlate with space metabolomics data; specific health anomalies experienced in microgravity; and determining how increasing metabolic activity is indicative of a causal effect between the existence of a microbial colony and/or a disease process.

Designing an efficient life support system can be important to maintain the minimum requirements for sustaining human life in space. This includes reliable rapid turnaround tools for detecting disease during spaceflight. Spaceflight is more complicated because the alterations in the gut microbiome are not well understood. Additional data may be collected by the system of the present invention to help determine issues that arise during spaceflight so that they can be avoided or alleviated. The system may track the microbiome of astronauts before, during, and after ISS missions. The Astronaut Microbiome project is evaluating culture-independent methods for studying the microbiome of astronauts, surfaces, and water on the ISS, largely using RNA and DNA metagenomic sequencing methods, which is timely and expensive.

Special benefits for astronauts may be realized by embodiments of the invention. High priority risks for deep space travel include increased radiation exposure, cognitive decline and behavioral conditions, inadequate food supply, unknown medication stability, and inflight medical conditions. Radiation can cause substantial damage to the microbiome. Cognitive and behavioral effects may also occur during spaceflight due to aberrations in the microbiome. Sterile meals and heavily filtered air reduce microbial diversity which can increase opportunistic infections. Currently, the recommended countermeasure to deal with bacterial infections in the ISS is the administration of antibiotics which may not be effective due to the increased virulence of bacteria in microgravity and may also induce dysbiosis with use. Additionally, probiotic treatment may result in unintended symptoms due to overuse creating imbalanced microbiomes. A rapid turnaround detection system, such as the system described herein, may offer a novel tool for studying the microbiome in space and mitigating these risks during long-duration spaceflight missions.

The present subject matter will now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Examples

The following EXAMPLES provide illustrative embodiments. Certain aspects of the following EXAMPLES are disclosed in terms of techniques and procedures found or contemplated by the present inventors to work well in the practice of the embodiments. In light of the present disclosure and the general level of skill in the art, those of skill will appreciate the following EXAMPLES are intended to be exemplary only and that numerous changes, modifications, and alterations can be employed without departing from the scope of the presently claimed subject matter.

Systems of the present invention can use a variety of analytical techniques to detect chemical compounds. Embodiments of the invention can employ an analytical technique called mass spectrometry to detect chemicals and/or chemical compounds collected through a collection device comprising a collection medium such as one or more sorbent tubes. The interior of the sorbent tube can comprise a material that traps chemicals as they are collected, typically, vacuumed, into or through the tube. The sorbent is chosen based on one or more compound of interest and is customized based on the target application.

A variety of sorbents can be used ranging from carbon alone or in combination with other sorbents to specialized polymers with applications spanning from breath analysis to air quality monitoring. Particular embodiments are directed to sorbents suitable for collecting and/or analyzing biosamples of a subject (human or animal) including urine, feces, saliva, breath, sputum and the like. See, e.g., FIGS. 1A-1C.

In some embodiments, a sorbent tube is connected through a quick-connect fitting attached to a toilet. Fecal VOCs are vacuumed from the headspace of the toilet into the sorbent tube which concentrates the VOCs. Once the chemicals are trapped, the tube is heated (either in place in the toilet or in a fixture connected to a remote chemical analyzer) to release the chemicals for analysis.

The chemical analyzer can be a variety of instruments such as a mass spectrometer, ion mobility spectrometer, and others. Sampling time can range from a few seconds to a couple of minutes. Compounds released are directed (e.g., vacuumed) into the chemical analyzer. The chemical analyzer draws in the sample from the sorbent tube. The compounds of interest are then analyzed by the chemical analyzer. An example chemical analyzer is a mass spectrometer such as, but not limited to, an MKS-RGA quadrupole mass spectrometer outlines an example overall detection process. A chemical finger print or mass spectrum is compiled and is referenced against a NIST (National Institute of Standards and Technology) mass spectra database of known compounds. The analysis system is configured to send a notice and/or alarm when a known compound is detected above a certain threshold. The analysis system is configured to track a chemical signature for each subject, such as a space crew member, over time.

The present invention may have sufficient sensitivity to detect relevant concentrations of clinically useful volatile biomarkers, detecting VOCs in the picogram to microgram concentration range. In some embodiments, the present invention may be used to test for the presence of viruses, such as, COVID-19. This sensitivity combined with the speed of analysis, simplified operation, low cost, portability, and low false alarm rate may allow a potential breakout opportunity to bring fast fecal analysis to the point-of-care in a new and unique way. It is believed that in some embodiments, a mass spectrometer can provide superior resolution compared to IMS. This device may be well suited for gas-phase analysis, and when complemented with an appropriate sample collection inlet (preconcentration tube, swab, or breathe tube), the system may offer a well-integrated point-of-care tool.

An example of specification for a Mass Spectrometer which can be used are as follows:

### MKS-RGA Analyzer Capabilities and Specifications:

Upgradeable and expandable threat detection library
Ethernet connection and USB Flash Drive
User-friendly EasyView™ software collects, displays and exports results in several formats
High-performance quadrupole mass spectrometer
Adaptable sample inlet system
Self-diagnoses of system failures with corrective actions
Sensitivity: ppt-ppm, depending on chemical
Response Time: 1 seconds, library dependent
Dynamic Range: >5 orders of magnitude
Dimensions: 18″ W×14.5″ L×21″ H (45 cm×36 cm×53 cm)
Weight: 40 lbs including preconcentrators
Power: 110 VAC/230 VAC, 50 Hz/60 Hz
Ionization: Non-radioactive, electron ionization, photoionization
Carry Gas: Ambient
Ion Polarity: Positive & Negative

High Resolution and Specificity.

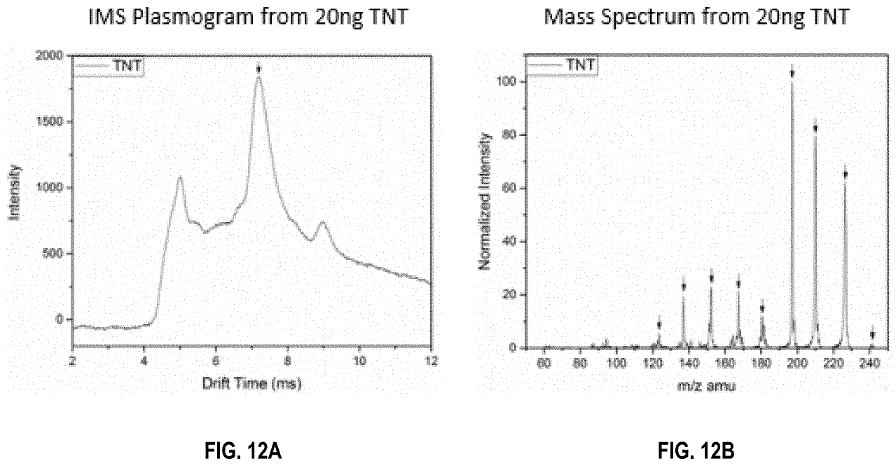

Much of the literature regarding VOC detection in stool samples uses ion mobility spectroscopy (IMS), which is the technique employed by electronic noses (E-nose). The system's mass spectrometer may provide higher resolution (specificity) than IMS, resulting in higher detection confidence. The figure here illustrates the far superior resolution of mass spectrometry compared to IMS. In FIGS. 12A-12B, IMS systems are commercially used for explosives detectors, and due to the low resolution of IMS systems, common perfumes are sometimes mistaken for actual explosives such as TNT which have similar chemical structures. Thus, false alarms are triggered. Here the mass spectrometer has a larger capacity for ions and higher resolution. More data points are available for extraction and identification of chemical compounds with mass spectrometry, lending itself to greater accuracy and reliability which is mandatory when evaluating the health of astronauts or patients.

Bacterial VOC Applications.

Bacterial VOC applications exist for breath analysis studies aimed to identify Staphylococcus aureus (SA) and Pseudomonas aeruginosa (PA) which are the common bacteria responsible for about half of all ventilator acquired pneumonia (VAP) and hospital acquired pneumonia (HAP) cases to allow for quicker initiation of antibiotic therapy. In short, SA and PA each produced VOC's that are not produced by humans and offered a unique set of target markers for identification of VAP and HAP as shown in Table 1.

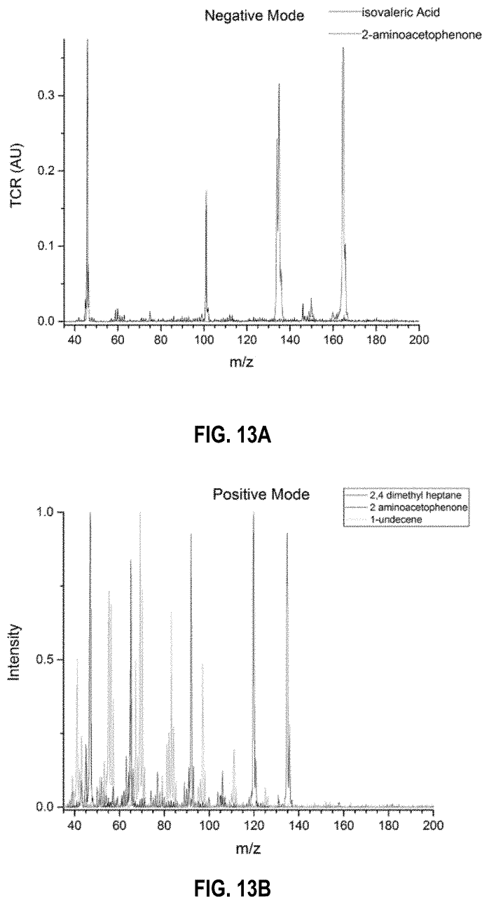

FIGS. 13A-13B illustrate detection of SA and PA VOCs with mass spectrometer in negative and positive modes. In FIGS. 13A-13B, isovaleric acid, 2-aminoacetophenone, 2,4 dimethyl-1-heptane, and 1-undecene can be differentiated. In some embodiments, a similar approach is used to build the fecal VOC library by data mining the literature for already established aggregate fecal VOC patterns in diseased and healthy stools.

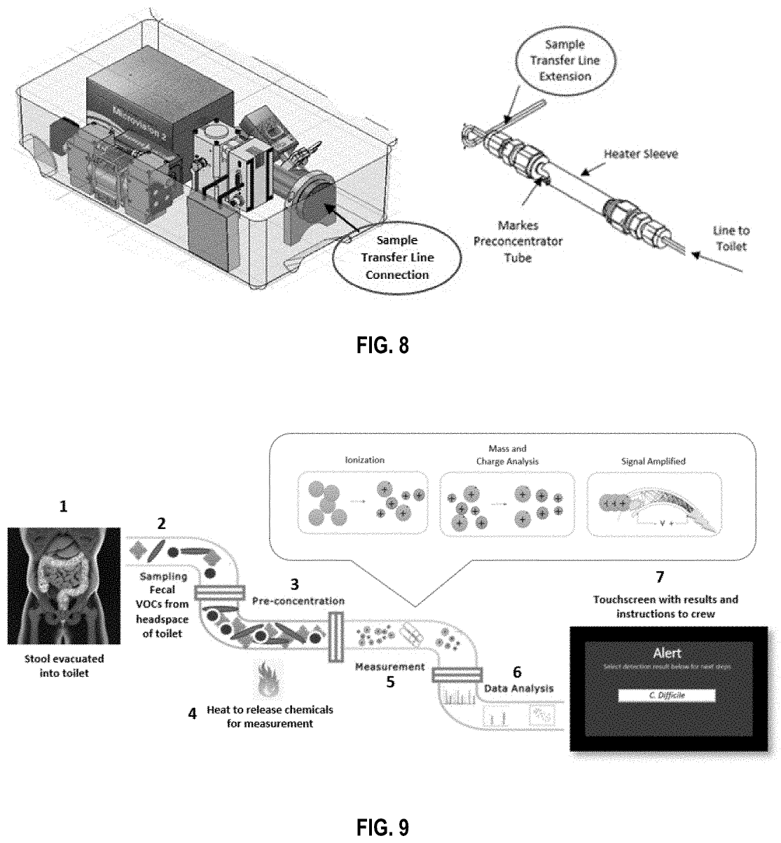

Preconcentrator (also known as sorbent) tubes allow large volumes of air to pass over a sorbent bed to collect and concentrate VOCs for analysis. This is beneficial for this application because the length of time spent using the lavatory facility varies and the sample may become diluted by air if directly sampled. Therefore, by concentrating the VOCs one can acquire more sample to deliver into the detection chamber at once, which can provide greater sensitivity and specificity during analysis and avoids losing VOCs that may be of low concentration. A thermal desorption preconcentration inlet is used to interface with the chemical analyzer and can integrate this technology with the MKS-RGA. In FIG. 8, an example Error! Reference source not found. configuration of a mass spectrometer (swab) inlet/transfer line connection is depicted. The sample transfer line to the sorbent tube assembly as shown is used in certain embodiments. The modular inlet design includes valves placed along a heated transfer line to direct air flow from the inlet selected into the analysis chamber, whether swab or sorbent tube (preconcentrator) (application dependent and selectable by user) and is easily configurable by the user from the software's user interface (UI).

An alternative embodiment may be to modify the sample collection bag to include a PDMS or other membrane port that can connect to the VOC sample transfer line without vacuuming particulates into the sorbent tube inlet. Upon completion of a sorbent tube inlet, the device can be used with an ISS toilet system to confirm appropriate connection and to determine the best VOC sample collection method.

Embodiments of the invention may control (programmatically) operations of the system such as operate a heater and control actuation of any valves or vacuum pumps as well as modifications to a user interface for selectable settings.

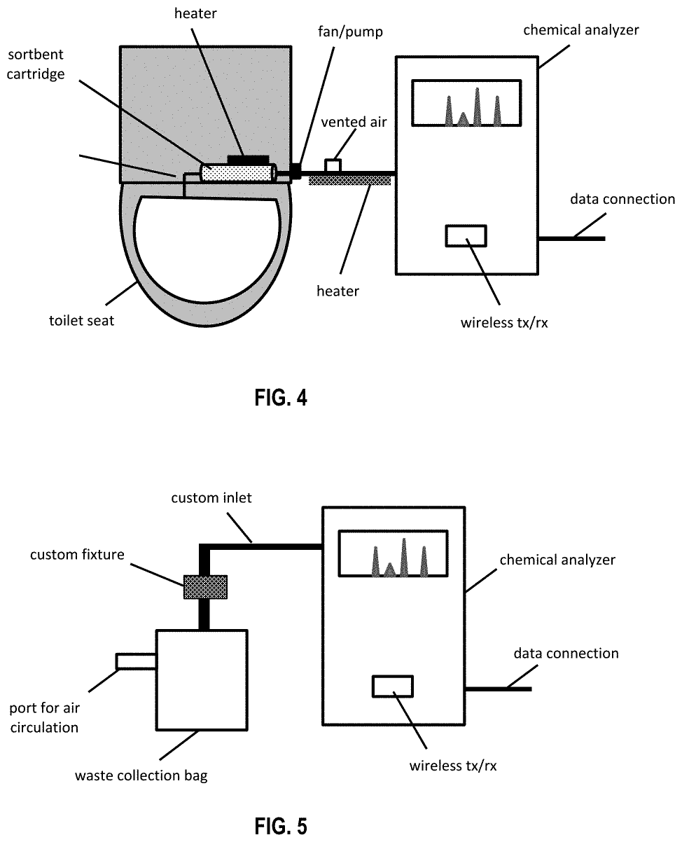

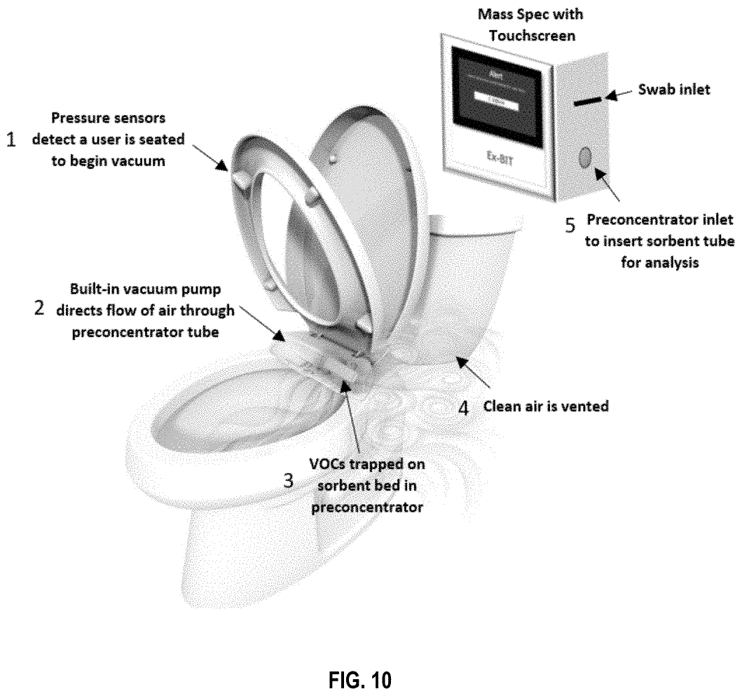

FIGS. 3A-3B illustrate the operation of an analysis system of the present invention comprising a toilet. Briefly, a small fan and/or vacuum pump can be incorporated into a compartment at the back of a standard toilet seat and the sorbent tube can be inserted into the compartment through an easy load and lock mechanism. Pressure or proximity sensors in or coupled to the toilet and/or the toilet seat can detect when a user is seated and activate the vacuum pump to begin evacuating the air from the headspace of the toilet bowl and through the sorbent tube. Fecal VOCs can be collected/trapped in the sorbent tube and clean air can be vented. As the user stands, the (pressure) sensors (in the seat) can direct the system to release to end the sample collection process (optionally closing/opening any valves). The sorbent tube can be removed (FIG. 3B) from the toilet (e.g., seat), typically placed in a biohazard package, and coupled to a preconcentrator inlet of the mass spectrometer. Finally, the inlet can be heated to release the VOCs from the sorbent bed of the preconcentrator tube into the detection chamber for analysis. FIG. 4 illustrates an embodiment that is similar to that in FIGS. 3A-3B. The main difference is that rather than removing the sorbent tube and placing it in a fixture, a heated transfer line connected the output of the sorbent tube to the input of a chemical analyzer. This provides for a hands free operation with faster results. FIG. 5 shows a waste collection bag (expandable/collapsible) connected to a fixture which draws the air into the chemical analyzer. The benefit of this embodiment is that pre-existing methods of stool collection can be maintained, without the use of a novel/customized toilet or toilet seat. Rather the bag can be used together with a fixture that draws the chemical sample from the bag into the chemical analyzer. FIG. 8 is an example analyzer with sample transfer line inlet connection and a preconcentrator tube. FIG. 9 is an example analysis system according to embodiments of the present invention. FIG. 10 is another example analysis system according to embodiments of the present invention. FIGS. 12A-12B are graphs illustrating IMS versus MS. FIGS. 13A-13B are graphs illustrating of negative and positive modes of detection.

While it is important to choose one or more sorbents to retain the VOCs of interest, it can also be important that the sorbent(s) release the VOCs during desorption. Thus, embodiments of the invention are configured to capture a wide range of VOCs, but do not want VOCs to irreversibly bind to the sorbent bed once preconcentrated. Therefore, specialty carbon sorbents can be selected based on the absorptive strength and ability to desorb the analyte. Analyte boiling point can be an important factor when deciding which sorbent coating to use in the preconcentrator tube.

Embodiments of the invention may use carboxen specialty sorbents as the sorbent class for fecal VOC detection. Carboxens have tapered pores, resulting in excellent thermodynamic properties for both adsorption and desorption. The surface chemistry of carboxen sorbents can also be tailored for specific target analytes, of which there are 19 different types of carboxen sorbents available commercially. Carboxen sorbents are one of the most efficient groups of adsorbents and are widely used in air sampling devices, purge traps, and gas chromatography columns because they provide excellent chromatography without a need for cryogenic cooling. Carboxens are also used for sampling very volatile compounds which are easily lost with other sorbents.

In some embodiments, data mining can be performed of the fecal microbiome literature to determine relevant VOCs to monitor. Chemical standards are obtained for the VOCs identified from the literature to analyze with the system of the present invention, particularly the VOCs from bacteria showing changes in microgravity and from opportunistic infections.

The chemical standards can be delivered to the system via a system that creates trace concentration levels of VOC mixtures using diffusion permeation tubes and a continuous gas stream. This method allows for controlled delivery of the gases at concentration levels expected for fecal VOCs and will provide limits of detection for the system. The data collected identifies the chemical fragmentation patterns for each volatile and determines desorption performances of the various sorbent bed types.

Analysis of fecal VOCs can be performed in real-time (while the subject is seated on the toilet or within 5 minutes thereafter) from the headspace of toilets within a home, care facility or hospital-setting using certain embodiments of the system. For example, patients testing positive for a bowel infection or bowel disease can be monitored using the system. In some embodiments, the patients may be tested for the present of viruses, for example, COVID-19. For example, in some instances, some patients suffering from COVID-19 experience digestive symptoms days before they experience respiratory distress. In some embodiments, the system of the present invention may be used to help detect these digestive symptoms, thereby allowing earlier detecting of the virus.

In some embodiments, (with informed consent from a patient), the toilet/toilet seat with the sample collection tube can be installed at a home, hospital, clinic, nursing home or other room of a patient.

All participants in a study can be given a non-identifiable code number and participant health information can be secured with encryption in a database (or locked in a filing cabinet within a locked office). Patient demographic data and general health information can be obtained from the medical records. Study participants can be asked to use the toilet as normal and practitioners can collect the sorbent tubes during their typical daily charting of the patient's stool. Sorbent tubes can be analyzed routinely (for example daily) for identification of aggregate VOC patterns among diseased and healthy stools using the system. When the analysis shows signs that the patient is experiencing a health event, then remedial measures can be taken to correct the issue, or additional tests can be done.

Special benefits for astronauts may be realized by embodiments of the invention. HRP high priority risks include radiation exposure, cognitive and behavioral conditions, inadequate food supply, medication stability, and inflight medical conditions. Radiation can cause substantial damage to the microbiome. Cognitive and behavioral effects may also occur during spaceflight due to aberrations in the microbiome. Sterile meals and heavily filtered air reduce microbial diversity which can increase opportunistic infections. Currently, the recommended countermeasure to deal with bacterial infections in the ISS is the administration of antibiotics which may not be effective due to the increased virulence of bacteria in microgravity and may also induce dysbiosis with use. A rapid turnaround detection system, such as the Ex-BIT™, may offer a novel tool for studying the microbiome in space and mitigating these risks during long-duration spaceflight missions.

In some embodiments, AI (Machine learning) can be used to determine correlations with diet/eating habits, environmental factors, behavioral data, and the VOC patterns discovered by the invention.

The sorbent tubes or embodiments of the invention are a special device that is different than a typical carbon filter that is only used to purify the air. Conventional filters have exposed carbon which also allows the user to touch the carbon during installation, which may leave oil residues on the filter from the skin-giving false results. Conventional filters also cannot typically be heated to a desired level (i.e., about 150° C. or higher) as the sorbent tube is formed of plastic material that, even if capable of heating, can off-gas its own VOCs once heated which can skew data. Also, the felt wrapping around the carbon filter may off gas the glue and the felt may burn and give off its own gas too. The carbon types typically used in conventional filters will most likely irreversibly bind chemicals given the purpose of prior filters was commonly to absorb odors, thus if it were capable of heating it would not release the captured chemicals into the system for analysis.

In some embodiments, the sorbent tubes are made of stainless steel. The body may be configured as a cylinder that can have an interior wall, layered and/or coated with a specialty sorbent so that the sorbent cannot be touched with oil from the fingers. The sorbent can be configured so that it will not irreversibly bind the chemicals of interest and will allow for analysis by the chemical analyzer.

For spaceflight purposes, the connection to the sorbent tube can be different from terrestrial uses. The ISS toilets typically use a plastic bag to collect the stool in the ISS toilet and there is a vacuum attached to it. One embodiment therefore comprise a similar bag that has a PDMS or other membrane to filter out any particulates (particularly if the astronaut has diarrhea) and another port on the other one end for airflow through the bag over the stool as a vacuum is applied. This is an alternative connection that can happen for use in space and may not be relevant to Earth.

In some embodiments, the sorbent tube can be loaded into the seat by sliding it through a guided canal in a heat block or a plastic unheated canal and can use a push and lock mechanism that is spring-loaded (push in once to lock in place, push a second time to spring it back out). The sorbent tube, once locked-in can be sealed to a small vacuum pump after it is pushed in and locked in place. This can direct air to flow over the sorbent material. Excess air can be vented.

For heating, some embodiments of the invention can use a small metal (e.g., aluminum) heat block/sleeve extending over at least a major, typically at least 80% or all the length of the sorbent tube. The toilet seat can be insulated/heat shielded. Heating can be controlled to initiate only after the user stands. The heat block can use a lever/latch push and lock mechanism to load the tube similar to the unheated version. It can be heated from ambient to 150° C. to release the volatiles collected.

To clean the tube, a bake-out program can heat the tube, while held in the toilet or toilet seat to a high temperature for a predetermined amount of time (e.g., 250° C.-300° C. for 10 minutes). There may be no requirement to change the tube out each time after a user uses the toilet. That is, the sorbent tube can be multi-use rather than single use-disposable.

Some embodiments use resistive heating with thermocouples to measure the temperature of the heater. The heating can be induction through a heater cartridge.

In some embodiments, the system is configured to detect that a person is sitting on the toilet seat. This can be done by a pressure sensor that is integrated in the rubber feet grips of the seat that trigger the fan once you sit down, and stop the fan/vacuum once you stand up. Other embodiments use light sensor, body heat sensor, opto-electronic sensor, a camera or a proximity sensor or combinations of same.

In some embodiments, the data provided by the chemical analyzer can be used in a variety of ways in different embodiments. AI algorithms using Convolutional Neural Networks (CNN) can be used for VOC analysis of the raw data. Independent of how the correlation data is generated, when a patient is using the toilet, the information can be used to track and/or adjust diet/eating habits, historical obesity data tracking, geographical mapping of disease incidence, food imports, domestic crops, pesticides, etc. for gastrointestinal health predictions.

Designing an efficient life support system can be important to maintain the minimum requirements for sustaining human life in space. This includes reliable rapid turnaround tools for detecting disease during spaceflight. Embodiments of the invention can use and/or contribute to a library of the VOCs emanating from the human body, known as the volatolome, for breath, saliva, blood, milk, skin, urine, and feces. Diseases related to these VOCs include colorectal cancer, Crohn's disease, ulcerative colitis, irritable bowel disease, Clostridium difficile, Rotovirus, Vibrio cholera, Campylobacter jejuni, and pelvic radiation toxicity. Spaceflight is more complicated because the alterations in the gut microbiome are not well understood. Additional data is collected by embodiments of the invention to help determine issues that arise during spaceflight so that they is avoided or alleviated.

The foregoing is illustrative of the present invention and is not to be construed as limiting thereof. Although exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

TABLE 1
VOC patterns emitted from samples of VAP and HAP

| Staphyloccocus aureus | Pseudomonas aeruginosa | Common to Both Pathogens |
|---|---|---|
| Isovaleric Acid (3-Methylbutanoic acid) | 1-undecene | Isopentanol (Isoamyl Alcohol) |
| Molecular formula: C5H10O2 | Molecular Formula: C11H22 | Formula: C5H12O |
| Molar mass: 102.13 g/mol | Molecular Weight: 154.29238 g/mol | Molar mass: 88.148 g/mol |
| 2-methyl-butanal | 2,4-dimethyl-1-heptane | Formaldehyde |
| Molecular Formula: C5H10O | Molecular Formula: C9H18 | Formula: CH2O |
| Average mass: 86.132 Da | Average mass: 126.239 Da | Molar mass: 30.031 g/mol |
| | 2-aminoacetophenone | Methyl Mercaptan (Methan Ethiol) |
| | Molecular Formula: C8H9NO | Formula: CH4S |
| | Molar mass: 135.16 g/mol | Molar mass: 48.11 g/mol |
| | 4-methyl-quinazoline | Trimethylamine |
| | Molecular Formula: C9H8N2 | Formula: C3H9N |
| | Average mass: 144.173 Da | Molar mass: 59.11 g/mol |
| | Hydrogen cyanide | |
| | Formula: HCN | |
| | Molar mass: 27.0253 g | |

That which is claimed is:

1. A gas collection and analysis system for detecting microbiomes, the system comprising:
a waste disposal site;
a sorbent cartridge coupled adjacent to the waste disposal site, wherein the sorbent cartridge includes a sorbent media configured to absorb a gas sample from within the waste disposal site;
a sorbent heating block configured to heat the sorbent cartridge and release the gas sample absorbed by the sorbent media; and
a pumping system coupling the sorbent cartridge to an ionized gas analyzer mass spectrometer, the pumping system configured to direct the flow of the released gas sample from the sorbent media to the gas analyzer mass spectrometer for chemical analysis.

2. The system of claim 1, further comprising one or more sensors coupled to the waste disposal site configured to control the pumping system and heating of the sorbent heating block.

3. The system of claim 1, wherein the ionized gas analyzer mass spectrometer comprises a quadrupole, an ion trap, a time of flight mass spectrometer, an ion mobility, a differential mobility, or a field asymmetric ion mobility spectrometer.

4. The system of claim 1, further comprising a controller configured to process data of the ionized gas analyzer mass spectrometer and/or concentration of absorbed and releases gases in real-time and over period of time.

5. The system of claim 1, wherein the ion mobility measurement may be done by IMS, DMS, FAIMS, or hyphenated techniques of ion mobility and mass spectrometry with gas chromatography.

6. The system of claim 1, wherein the ionization mechanism comprises nickel-63 foil, electron ionization, corona discharge, chemical ionization, photoionization, electro-spray, atmospheric pressure chemical ionization, thermal ionization, DART, desorption electro spray, or metal spray.

7. The system of claim 1, wherein the sorbent cartridge is reusable, thereby allowing the system to obtain gas samples of multiple, different users.

8. The system of claim 1, wherein the sorbent cartridge comprises a carboxen sorbent, a liquid sorbent, or one or more membranes.

9. The system of claim 1, wherein the sorbent cartridge comprises a single or multiple sorbent media for analysis.

10. The system of claim 1, wherein the waste disposal site is a toilet.

11. The system of claim 1, wherein the waste disposal site is a waste collection bag.

12. The system of claim 1, wherein with the sorbent cartridge is configured to be attached to a disposable mouthpiece to obtain the gas sample from a breath analysis.

13. The system of claim 1, wherein the gas sample is utilized for online analysis via gas chromatography.

14. The system of claim 1, wherein the ionized gas analyzer mass spectrometer utilizes gas chromatography.

15. The system of claim 1, wherein the system is configured to monitor and track the gas samples of a user for evaluation.

16. A method for detecting microbiomes of a user, the method comprising:
providing a gas collection and analysis system comprising a waste disposal site, wherein the waste disposal site, a sorbent cartridge coupled adjacent to the waste disposal site, wherein the sorbent cartridge includes a sorbent media configured to absorb a gas sample from within the waste disposal site, a sorbent heating block configured to heat the sorbent cartridge and release the gas sample absorbed by the sorbent media, and a pumping system coupling the sorbent cartridge to an ionized gas analyzer mass spectrometer, the pumping system configured to direct the flow of the released gas sample from the sorbent media to the gas analyzer mass spectrometer for chemical analysis;
having the user sit on the waste disposal site;
absorbing a gas sample of the user from the waste disposal site into the sorbent cartridge;
having the user stand up from the waste disposal site;
heating the sorbet cartridge to release a gas sample from the sorbent cartridge;
pumping the released gas sample from the sorbent cartridge to the gas analyzer mass spectrometer;
determining the chemical compounds in the gas sample based on a chemical analysis from the gas analyzer; and
providing the user health data based on the chemical analysis.

17. The method of claim 17, comprising removing the sorbent cartridge from the waste disposal site for analysis at a remote location.

18. The method of claim 17, wherein the gas collection and analysis system further comprises one or more sensors coupled to the waste disposal site, the sensors are configured to control the pumping system and heating of the sorbent heating bloc when the user stands up from the waste disposal site.

19. The method of claim 17, wherein the gas collection and analysis system further comprises a controller configured to process data of the ionized gas analyzer mass spectrometer and/or concentration of absorbed and releases gases to the user in real-time and over period of time.

20. The method of claim 17, where the sorbent cartridge is reusable, thereby allowing the system to obtain gas samples of multiple, different users.
